(19) United States
(12) Patent Application Publication
(10) Pub. No.: US 2024/0009328 A1
(43) Pub. Date: Jan. 11, 2024

(54) RATIONAL DESIGN OF THREE-DIMENSIONAL BIODEGRADABLE CORE-UPCONVERSION TETRAGONAL NANODENDRITES WITH ULTRABRIGHT LUMINESCENCE FOR VARIOUS BIOMEDICAL APPLICATIONS

(71) Applicant: RESEARCH & BUSINESS FOUNDATION SUNGKYUNKWAN UNIVERSITY, Suwon-si (KR)

(72) Inventors: Donghwan KIM, Suwon-si (KR); Murad ABUALREJAL, Suwon-si (KR)

(73) Assignee: RESEARCH & BUSINESS FOUNDATION SUNGKYUNKWAN UNIVERSITY, Suwon-si (KR)

(21) Appl. No.: 18/219,922

(22) Filed: Jul. 10, 2023

(30) Foreign Application Priority Data
Jul. 11, 2022 (KR) 10-2022-0084750

(51) Int. Cl. A61K 49/00; B82Y 5/00

(52) U.S. Cl. CPC A61K 49/0065; B82Y 5/00

(57) ABSTRACT

The present disclosure relates to an upconversion nanoparticle, which is represented by the following Chemical Formula 1 and comprises a nanoparticle doped with lanthanide ion:

Li₃ZrF₇:Ln³⁺[ Chemical Formula 1]

where Ln is a lanthanide element selected from the group consisting of La, Ce, Pr, Nd, Pm, Sm, Eu, Gd, Tb, Dy, Ho, Er, Tm, Yb, Lu, and combinations thereof.

### CROSS REFERENCE TO RELATED APPLICATION

The present disclosure claims the priority of Korean Patent Application No. 10-2022-0084750, filed on Jul. 11, 2022, the disclosures of which are incorporated herein by reference for all purposes.

### BACKGROUND OF THE INVENTION

### 1. Field of the Invention

The present disclosure relates to upconversion nanoparticles and method for their preparation.

### 2. Description of the Related Art

Upconversion nanoparticles (UCNPs) doped with lanthanide element (Ln 3+) are very promising materials for applications such as chemotherapy, drug delivery, photothermal therapy, photodynamic therapy, biosensing and bioassays. The unique photoluminescent properties of upconversion nanoparticles lead to excellent visible light absorption, emission bandwidth, photobleaching resistance, low noise, stable light scattering and zero autofluorescence background.

Recently, interesting research on upconversion nanoparticles has been actively conducted in various biomedical applications, and upconversion nanoparticles exhibit unique advantages such as unique high resolution, minimal background interference, negligible damage to living organisms, and deep tissue excitation and detection of in vivo fluorescent signals. In addition, upconversion nanoparticles designed with a small size of less than 5.5 nm can be degraded and are a very ideal material required for kidney clearance. In addition, the harmless removal of upconversion nanoparticles injected into the body after performing their diagnostic or therapeutic function within an appropriate time is essential.

However, K3ZrF7:Yb/Er, a previously reported biodegradable upconversion nanoparticle has a problem in that the biodegradation time is only 8 hours, so there is a limit to practical use for diagnosis or treatment, and the upconversion luminous efficiency is very low.

Therefore, there is a need to develop biodegradable upconversion nanoparticles having a long biodegradation time and high upconversion luminous efficiency.

Korean Patent Publication No. 10-2022-0027648 relates to an upconversion nanophosphor capable of various color adjustments, and discloses a tetragonal upconversion nanophosphor having a core/multiple shell structure, which can emit blue light, green light, red light, and combinations thereof when excited with near infrared light having wavelengths of 800±20 nm, 980±20 nm, and 1532±20 nm. However, the above patent does not disclose biodegradation, and the material constituting the upconversion nanophosphor is also different from the present disclosure.

### SUMMARY OF THE INVENTION

The present disclosure is to solve the problems of the prior art described above, and an object of the present disclosure is to provide upconversion nanoparticles.

In addition, an object of the present disclosure is to provide a method for preparing the upconversion nanoparticles.

In addition, an object of the present disclosure is to provide a bioimaging probe including the upconversion nanoparticles.

However, the technical objects to be achieved by the embodiments of the present disclosure are not limited to the technical objects described above, and other technical objects may exist.

In order to achieve the above technical object, a first aspect of the present disclosure provides a upconversion nanoparticle which is represented by the following Chemical Formula 1 and comprises a nanoparticle doped with lanthanide ion:

Li₃ZrF₇:Ln³⁺ [Chemical Formula 1]

(In Chemical Formula 1, Ln is a lanthanide element selected from the group consisting of La, Ce, Pr, Nd, Pm, Sm, Eu, Gd, Tb, Dy, Ho, Er, Tm, Yb, Lu, and combinations thereof).

According to an embodiment of the present disclosure, the nanoparticle doped with the lanthanide ion may be the nanoparticle doped with two or more different lanthanide ions, but is not limited thereto.

According to an embodiment of the present disclosure, the nanoparticle doped with the lanthanide ion may include Li₃ZrF₇:Er³⁺, Yb³⁺, but is not limited thereto.

According to an embodiment of the present disclosure, the upconversion nanoparticle may be biodegradable, but is not limited thereto.

According to an embodiment of the present disclosure, the upconversion nanoparticle may have an upconversion luminescence (UCL) intensity of 5×10⁵ or more in a wavelength range of 400 nm to 700 nm, but is not limited thereto.

In addition, a second aspect of the present disclosure provides a method for preparing an upconversion nanoparticle, comprising preparing a mixture by dissolving a lithium salt, a zirconium salt, and a lanthanide ion precursor in an organic solvent; and heating the mixture.

According to an embodiment of the present disclosure, the lanthanide ion precursor may comprise two or more different lanthanide ion precursors, but is not limited thereto.

According to an embodiment of the present disclosure, the lanthanide ion precursor may comprise one represented by Chemical Formula 2 below, but is not limited thereto:

Ln(CH₃CO₂)·4H₂O [Chemical Formula 2]

(In Chemical Formula 2, Ln is La, Ce, Pr, Nd, Pm, Sm, Eu, Gd, Tb, Dy, Ho, Er, Tm, Yb or Lu).

According to an embodiment of the present disclosure, the lithium salt may include one selected from the group consisting of lithium trifluoroacetate, lithium tetrafluoroborate, lithium hexafluorophosphate, lithium bis(trifluoromethylsulfonyl)imide, lithium trifluoromethane sulfonate, lithium acetate, lithium nitrate, lithium perchlorate, lithium hexafluoroarsinate, lithium bis(pentafluoroethylsulfonyl)imide, lithium dicyanamide, lithium tetrachloroaluminate, lithium hexafluoroantimonate, and combinations thereof, but is not limited thereto.

According to an embodiment of the present disclosure, the zirconium salt may include one selected from the group consisting of zirconium (IV) acetylacetonate, zirconium acrylate, zirconium (IV) bis(diethyl citrato)dipropoxide, zirconium bromonorbornenelactone carboxylate triacrylate, zirconium (IV) butoxide, zirconium (IV) tert-butoxide, zirconium (IV) carbonate basic, zirconium carboxyethylacrylate (in n-propanol), zirconium (IV) chloride tetrahydrofuran complex, zirconium (IV) ethoxide, zirconium (IV) isopropoxide isopropanol complex, zirconium (IV) propoxide solution (in N-propyl alcohol), zirconium (IV) 2,2,6,6-tetramethyl-3,5-heptanedionate, zirconium (IV) trifluoroacetylacetonate, and combinations thereof, but is not limited thereto.

According to an embodiment of the present disclosure, the organic solvent may include one selected from the group consisting of 1-octadecene, 1-nonadecene, cis-2-methyl-7-octadecene, 1-heptadecene, 1-hexadecene, 1-pentadecene, 1-tetradecene, 1-tridecene, 1-undecene, 1-dodecene, 1-decene, and combinations thereof, but is not limited thereto.

According to an embodiment of the present disclosure, the fatty acid may include one selected from the group consisting of oleic acid, elaidic acid, gondoic acid, erucic acid, nervonic acid, eicosadienoic acid, mead acid, and combinations thereof, but is not limited thereto.

According to an embodiment of the present disclosure, the heating may be performed at a temperature range of 100° C. to 350° C., but is not limited thereto.

In addition, a third aspect of the present disclosure provides a bioimaging probe comprising the upconversion nanoparticle according to the first aspect of the present disclosure.

The above-mentioned solutions are merely exemplary and should not be construed as limiting the present disclosure. In addition to the above-described exemplary embodiments, additional embodiments may exist in the drawings and detailed description of the disclosure.

### BRIEF DESCRIPTION OF THE DRAWINGS

FIG. 1 is a flowchart of a method for preparing an upconversion nanoparticle according to an embodiment of the present disclosure.

FIG. 2 is a synthesis mechanism of upconversion nanoparticles according to an embodiment of the present disclosure.

FIG. 3 is an HRTEM image of upconversion nanoparticles according to an embodiment of the present disclosure.

FIG. 4 is an HRTEM image of upconversion nanoparticles according to Comparative Example 1 of the present disclosure.

FIG. 5 is an HRTEM image of upconversion nanoparticles according to Comparative Example 2 of the present disclosure.

FIG. 6 is a HADDF-STEM image and elemental mapping analysis result of upconversion nanoparticles according to an embodiment of the present disclosure.

FIG. 7 is an energy dispersive x-ray spectroscopy (EDS) spectrum of upconversion nanoparticles according to an embodiment of the present disclosure.

FIG. 8 is an (a) before degradation and (b) after degradation of upconversion nanoparticles according to an embodiment of the present disclosure.

FIG. 9 is an XPS spectrum of upconversion nanoparticles according to an embodiment of the present disclosure.

FIG. 10 is a result of measuring a change in brightness of upconversion luminescence over time of upconversion nanoparticles according to an embodiment of the present disclosure.

FIG. 11 is a UCL spectrum of upconversion nanoparticles according to an Example and Comparative Example of the present disclosure.

In FIG. 12, (A) and (B) are graphs showing measurement of UCL intensity of upconversion nanoparticles according to an embodiment of the present disclosure in various solvents, and (C) is a graph showing measurement of ion release.

In FIG. 13, (A) and (B) are HRTEM images before and after degradation of upconversion nanoparticles according to an embodiment of the present disclosure, and (C) and (D) are SEAD patterns of upconversion nanoparticles according to an embodiment of the present disclosure.

FIG. 14 is an HRTEM image of upconversion nanoparticles coated with silica before and after degradation of the nanoparticles according to an embodiment of the present disclosure.

FIG. 15 is a HADDF-STEM image and elemental mapping analysis result of upconversion nanoparticles coated with silica according to an embodiment of the present disclosure.

FIG. 16 is an energy dispersive x-ray spectroscopy (EDS) spectrum of nanoparticles coated with silica according to an embodiment of the present disclosure.

### DETAILED DESCRIPTION OF THE INVENTION

The embodiments of the present disclosure will be described in detail with reference to the accompanying drawings to the extent that a person having ordinary skill in the art can easily understand.

It is obvious that the present disclosure can be implemented in various forms, and is not limited to the embodiments disclosed herein. The descriptions on the components which are not directly related with the descriptions of the present disclosure will be omitted for the sake of clear understanding of the present disclosure. Similar components are given similar reference numbers throughout the specification.

Throughout the specification, the phrase that a component is “connected to” another component means that the component is “directly connected” to the component or the component is “electrically connected” to the component through another component.

Throughout the specification, the phrase that a component is mounted “on”, “upper”, “above”, “under”, “lower”, “below” another component means that the component is contacting with the component, or another component may be interposed between the above-mentioned two components.

Throughout the specification, the phrase that a component “comprises” another component means that unless otherwise stated, the component may further comprise another component, not excluding other components.

The terms “about”, substantially”, etc. used throughout the specification means that when a natural manufacturing and a substance allowable error are suggested, such an allowable error corresponds the value or is similar to the value, and such values are intended for the sake of clear understanding of the present disclosure or to prevent an unconscious infringer from illegally using the present disclosure. The terms “a step wherein-” or a “step of-” does not mean “a step for the sake of-”.

Throughout the specification, the term “a combination thereof” recited in the expression of the Markush type claim means that at least one or more mixing or combination may be selected from a group consisting of multiple components recited in the expression of the Markush type, more specifically, it means that one or more components selected from a group consisting of components can be included.

Throughout the specification, reference to “A and/or B” means “A or B, or A and B”.

Hereinafter, the upconversion nanoparticles and their preparation method will be described in detail with reference to embodiments, examples and drawings. However, the present disclosure is not limited to these embodiments and examples and drawings.

As a technical means for achieving the above technical objects, a first aspect of the present disclosure provides a upconversion nanoparticle which is represented by the following Chemical Formula 1 and comprises a nanoparticle doped with lanthanide ion:

Li₃ZrF₇:Ln³⁺ [Chemical Formula 1]

(In Chemical Formula 1, Ln is a lanthanide element selected from the group consisting of La, Ce, Pr, Nd, Pm, Sm, Eu, Gd, Tb, Dy, Ho, Er, Tm, Yb, Lu, and combinations thereof).

The upconversion phenomenon is a phenomenon in which photons with lower energy are continuously absorbed and photons with higher energy are emitted. The upconversion can be applied to various application fields, such as converting infrared light, which is abundant in sunlight, into visible light to increase the efficiency of solar cells, or converting infrared light with high biological permeability into visible light inside brain tissue to provide effective photo stimulation. However, conventional upconversion nanoparticles have limitations in that upconversion efficiency and spectral purity are low, and have a short biodegradation time of about 12 hours or less.

On the other hand, the upconversion nanoparticles according to the present disclosure have an increased biodegradation period from several hours to several days, and have an upconversion luminescence intensity that is about 300 times higher than that of last reported biodegradable upconversion. Accordingly, the upconversion nanoparticles according to the present disclosure can be applied to various biomedical fields such as bioimaging and used more practically.

According to an embodiment of the present disclosure, the nanoparticles doped with lanthanide ions may be the nanoparticles doped with two or more different lanthanide ions, but are not limited thereto.

The upconversion nanoparticles according to the present disclosure may be doped with one kind of lanthanide ions, or may be doped with two or more different lanthanide ions. The type of lanthanide ion may be selected and doped according to the environment and purpose in which the upconversion nanoparticles are to be used.

According to an embodiment of the present disclosure, the nanoparticles doped with lanthanide ions may include Li₃ZrF₇:Er³⁺, Yb³⁺, but are not limited thereto.

According to an embodiment of the present disclosure, the upconversion nanoparticles may be biodegradable, but are not limited thereto.

Conventional biodegradable upconversion nanoparticles are degraded within about 12 hours after being introduced into the body, so there is a disadvantage that the period is too short for diagnosis and treatment. The upconversion nanoparticles according to the present disclosure can be used more efficiently in diagnosis and treatment by increasing the biodegradation period from several days to several tens of days.

According to an embodiment of the present disclosure, the upconversion nanoparticles may have an upconversion luminescence (UCL) intensity of 5×10⁵ or more in a wavelength range of 400 nm to 700 nm, but are not limited thereto.

An upconversion phenomenon, which is a phenomenon in which photons having lower energy are continuously absorbed and photons having higher energy are emitted, occurs in lanthanide ions (Ln³⁺) doped in an inorganic host material. As a result of complex interactions between the host material and the lanthanide ions doped in the host material, upconversion efficiency and emission spectrum may be determined.

The upconversion nanoparticles according to the present disclosure use Li₃ZrF₇ as an inorganic host material, and improve upconversion efficiency and spectrum purity compared to conventional upconversion nanoparticles by selecting the type of lanthanide ion and doping the selected lanthanide in the host material.

Specifically, since the upconversion nanoparticles according to the present disclosure have an upconversion luminescence intensity that is about 300 times higher than that of last reported biodegradable upconversion, they can be applied to application fields and show more excellent effects.

In addition, a second aspect of the present disclosure provides a method for preparing an upconversion nanoparticle, comprising preparing a mixture by dissolving a lithium salt, a zirconium salt, and a lanthanide ion precursor in an organic solvent; and heating the mixture.

With respect to the method for manufacturing an upconversion nanoparticle according to the second aspect of the present disclosure, detailed descriptions overlapping with the descriptions of the first aspect of the present disclosure are omitted, but even if the description is omitted, the contents described in the first aspect of the present disclosure may be equally applied to the second aspect of the present disclosure.

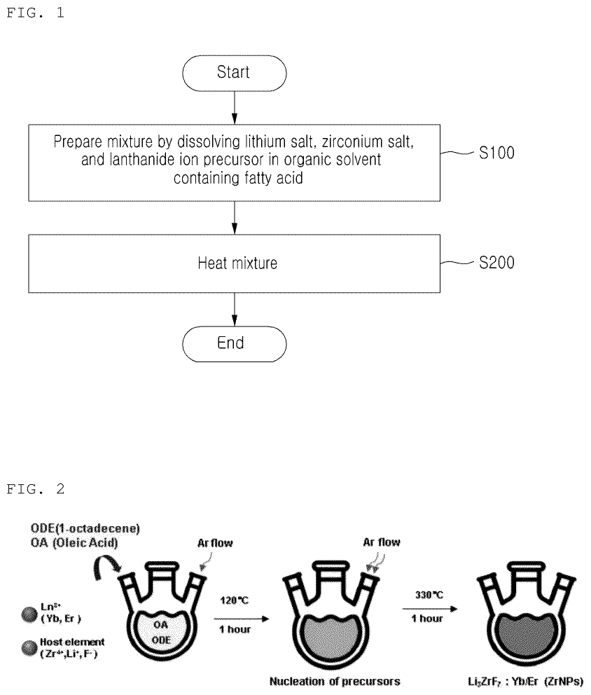

FIG. 1 is a flowchart of a method for preparing an upconversion nanoparticle according to an embodiment of the present disclosure.

First, a mixture is prepared by dissolving a lithium salt, a zirconium salt, and a lanthanide ion precursor in an organic solvent containing a fatty acid S100.

According to an embodiment of the present disclosure, the lanthanide ion precursor may include two or more different lanthanide ion precursors, but is not limited thereto.

As described above, the upconversion nanoparticles according to the present disclosure may be doped with one kind of lanthanide ion or two or more different lanthanide ions, which can be controlled by selecting the type of the lanthanide ion precursor added in the step of preparing the mixture S100.

According to an embodiment of the present disclosure, the lanthanide ion precursor may include one represented by Chemical Formula 2 below, but is not limited thereto:

Ln(CH₃CO₂)·4H₂O [Chemical Formula 2]

(In Chemical Formula 2, Ln is La, Ce, Pr, Nd, Pm, Sm, Eu, Gd, Tb, Dy, Ho, Er, Tm, Yb or Lu).

According to an embodiment of the present disclosure, the lithium salt may include one selected from the group consisting of lithium trifluoroacetate, lithium tetrafluoroborate, lithium hexafluorophosphate, lithium bis(trifluoromethylsulfonyl)imide, lithium trifluoromethane sulfonate, lithium acetate, lithium nitrate, lithium perchlorate, lithium hexafluoroarsinate, lithium bis(pentafluoroethylsulfonyl)imide, lithium dicyanamide, lithium tetrachloroaluminate, lithium hexafluoroantimonate, and combinations thereof, but is not limited thereto.

According to an embodiment of the present disclosure, the zirconium salt may include one selected from the group consisting of zirconium (IV) acetylacetonate, zirconium acrylate, zirconium (IV) bis(diethyl citrato)dipropoxide, zirconium bromonorbornenelactone carboxylate triacrylate, zirconium (IV) butoxide, zirconium (IV) tert-butoxide, zirconium (IV) carbonate basic, zirconium carboxyethylacrylate (in n-propanol), zirconium (IV) chloride tetrahydrofuran complex, zirconium (IV) ethoxide, zirconium (IV) isopropoxide isopropanol complex, zirconium (IV) propoxide solution (in N-propyl alcohol), zirconium (IV) 2,2,6,6-tetramethyl-3,5-heptanedionate, zirconium (IV) trifluoroacetylacetonate, and combinations thereof, but is not limited thereto.

According to an embodiment of the present disclosure, the organic solvent may include one selected from the group consisting of 1-octadecene, 1-nonadecene, cis-2-methyl-7-octadecene, 1-heptadecene, 1-hexadecene, 1-pentadecene, 1-tetradecene, 1-tridecene, 1-undecene, 1-dodecene, 1-decene, and combinations thereof, but is not limited thereto.

According to an embodiment of the present disclosure, the fatty acid may include one selected from the group consisting of oleic acid, elaidic acid, gondoic acid, erucic acid, nervonic acid, eicosadienoic acid, mead acid, and combinations thereof, but is not limited thereto.

Then, the mixture is heated S200.

According to an embodiment of the present disclosure, the heating may be performed at a temperature range of 100° C. to 350° C., but is not limited thereto.

In addition, a third aspect of the present disclosure provides a bioimaging probe comprising the upconversion nanoparticle according to the first aspect of the present disclosure.

With respect to the bioimaging probe according to the third aspect of the present disclosure, detailed descriptions overlapping with the descriptions of the first aspect and/or second aspect of the present disclosure are omitted, but even if the description is omitted, the contents described in the first aspect and/or second aspect of the present disclosure may be equally applied to the third aspect of the present disclosure.

Unlike last reported biodegradable upconversion nanoparticles that were biodegraded within about 12 hours, the upconversion nanoparticles according to the present disclosure have increased biodegradation period from hours to several days, and have an upconversion luminescence intensity that is about 300 times higher than that of last reported biodegradable upconversion. Accordingly, the upconversion nanoparticles according to the present disclosure can be applied to various biomedical fields such as bioimaging and can be used more practically.

The present invention will be described in more detail through the following examples, but the following examples are for illustrative purposes only and are not intended to limit the scope of the present disclosure.

### [Example] Li₃Zrf₇:Er³⁺, Yb³⁺

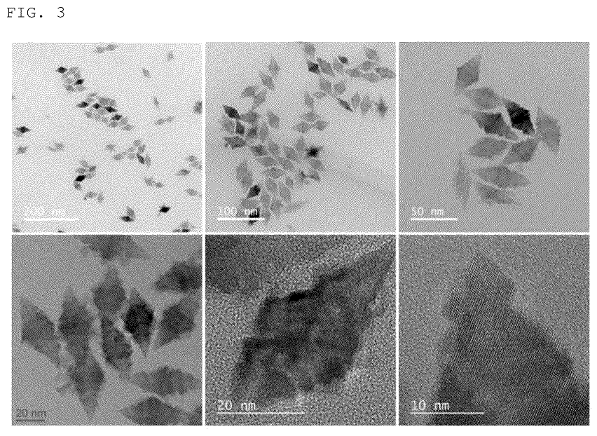

FIG. 2 is a synthesis mechanism of upconversion nanoparticles according to an embodiment of the present disclosure.

First, stoichiometric amounts of zirconium (IV) acetylacetonate, Yb(CH₃CO₂)·4H₂O, Er(CH₃CO₂)·4H₂O mixed with 2.5 mmol of lithium trifluoroacetate were dissolved in 20 ml of 1-octadecene and 20 ml of oleic acid, while vigorously stirred in a 250 ml three-neck flask.

The mixture was then heated to 110° C. for 30 minutes under vacuum, and after the flask was charged with Ar, the temperature of the solution was gradually raised to 330° C. and held at this temperature for 1 hour.

After cooling the solution to room temperature under ambient conditions, the prepared upconversion nanoparticles, Li₃ZrF₇:Er³⁺, Yb³⁺, were collected by centrifugation (10,000 rpm for 10 min) and washed with ethanol (10 mL, 3 times).

The prepared Li₃ZrF₇:Er³⁺, Yb³⁺ was redispersed in cyclohexane (10 mL) for further experiments and characterization.

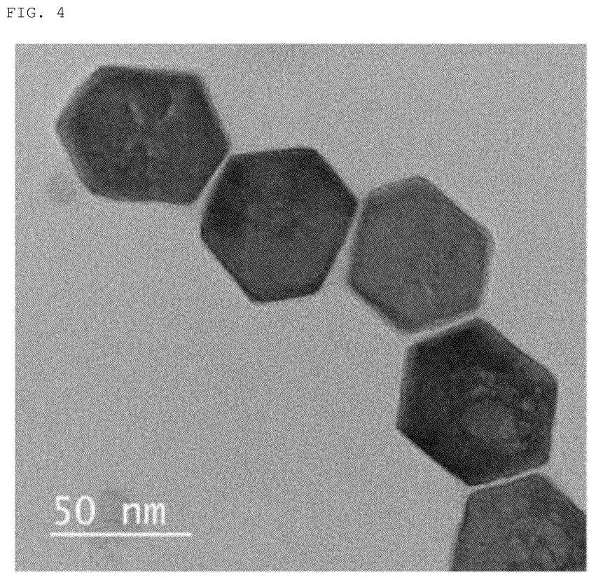

FIG. 3 is an HRTEM image of upconversion nanoparticles according to an embodiment of the present disclosure.

Referring to FIG. 3, it can be confirmed that the upconversion nanoparticles according to the present disclosure were prepared in high yield and high purity without formation of unwanted by-products, and were uniformly monodispersed, and had an average diameter of 50±5 nm.

### [Comparative Example 1] K₃ZrF₇:Er³⁺, Yb³⁺

Through the method disclosed in the paper (Binbin Ding et al., Biodegradable Upconversion Nanoparticles Induce Pyroptosis for Cancer Immunotherapy, Nano letter, 2021, 21, 8281-8289), biodegradable upconversion nanoparticles, K₃ZrF₇:Er³⁺, Yb³⁺ were prepared and used as Comparative Example 1.

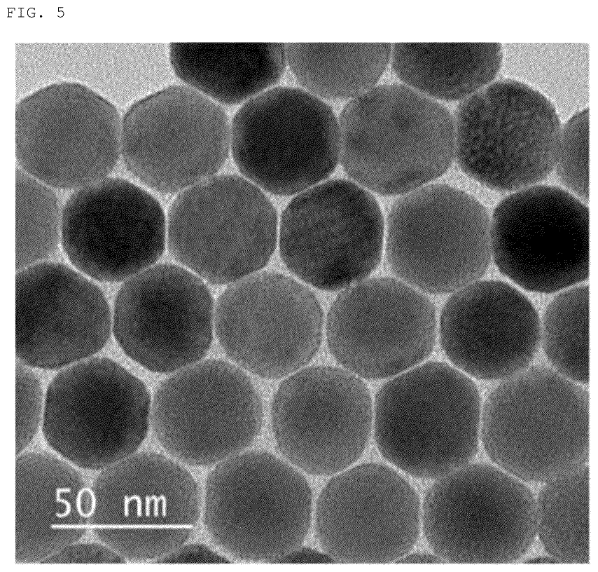

FIG. 4 is an HRTEM image of upconversion nanoparticles according to Comparative Example 1 of the present disclosure.

### [Comparative Example 2] NaYF₄: Er³⁺, Yb³⁺

Through the method disclosed in the paper (R. Arppe et al., Quenching of the upconversion luminescence of NaYF₄: Yb³⁺, Er³⁺ and NaYF₄: Yb³⁺, Tm³⁺ nanophosphors by water: the role of the sensitizer Yb³⁺ in non-radiative relaxation, Nanoscale, 7 (2015) 11746-11757), conventional biodegradable upconversion nanoparticles, NaYF₄: Er³⁺, Yb³⁺ were prepared and used as Comparative Example 2.

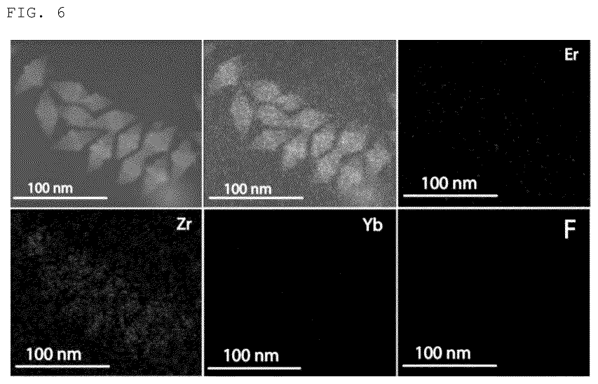

FIG. 5 is an HRTEM image of upconversion nanoparticles according to Comparative Example 2 of the present disclosure.

### [Experimental Example 1] Characterization of Example (Li₃ZrF₇:Er³⁺, Yb³⁺)

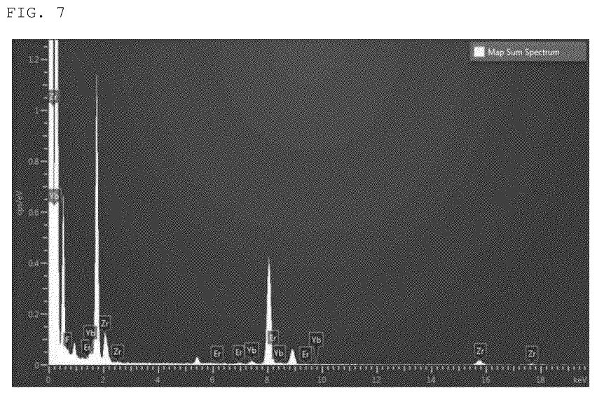

FIG. 6 is a HADDF-STEM image and elemental mapping analysis result of upconversion nanoparticles according to an embodiment of the present disclosure.

Referring to FIG. 6, it can be confirmed that the upconversion nanoparticles according to the present disclosure show a core nanodendritic shape.

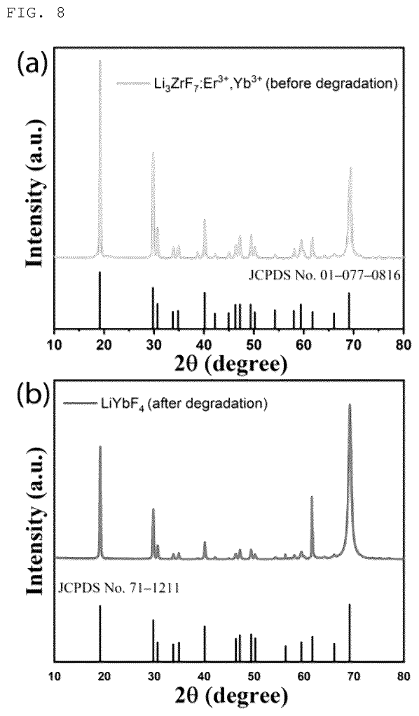

FIG. 7 is an energy dispersive x-ray spectroscopy (EDS) spectrum of upconversion nanoparticles according to an embodiment of the present disclosure.

The energy dispersive X-ray Spectroscopy (EDS) of upconversion nanoparticles, which is composed of elements Zr, Yb, Er and F and has three-dimensional core Li₃ZrF₇:Er³⁺,Yb³⁺ biodegradable nanodendritic crystal, can be confirmed with reference to FIG. 7.

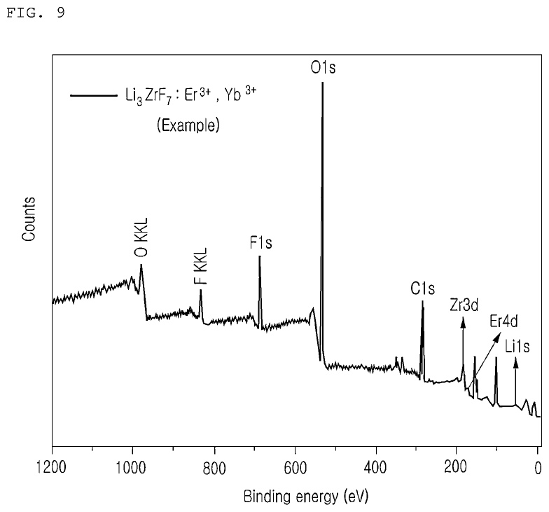

FIG. 8 is an upconversion nanodendrites(UCND) (a) before degradation and (b) after degradation of upconversion nanoparticles according to an embodiment of the present disclosure.

Referring to FIG. 8, (a) the upconversion nanoparticles of Example (Li₃ZrF₇:Er³⁺,Yb³⁺) were indexed according to the tetragonal phase standard pattern (JCPDS No. 01-077-0816), and through this, it was confirmed that there were no impurities in the upconversion nanoparticles of Example. (b) XRD pattern of the present disclosure after water degradation for 20 days, which could be well-indexed to LiYbF₄ (JCPDS File No. 71-1211).

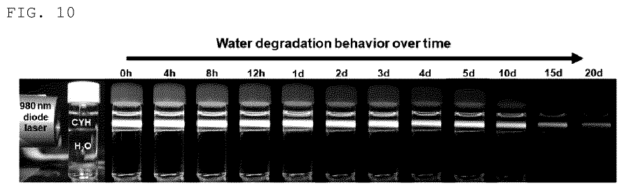

FIG. 9 is an XPS spectrum of upconversion nanoparticles according to an embodiment of the present disclosure.

Referring to FIG. 9, it was confirmed that Li is, Er 4d, Zr 3d, Yb 4d, C is, O is, and F is peaks existed. The combination of Li, Zr, Er and Yb is essential to enhance NIR absorption, maximize UCL efficiency and reduce energy extinction.

### [Experimental Example 2] Biodegradability Evaluation Experiment

An experiment was conducted to evaluate the biodegradability of the biodegradable upconversion nanoparticles according to an embodiment. Specifically, the upconversion nanoparticles (Li₃ZrF₇:Er³⁺,Yb³⁺) prepared in Example were dispersed in cyclohexane/water (1 ml/3 ml), and excited by a 980 nm diode laser, so that a change in brightness of upconversion luminescence of Example was measured at the interface of cyclohexane/water.

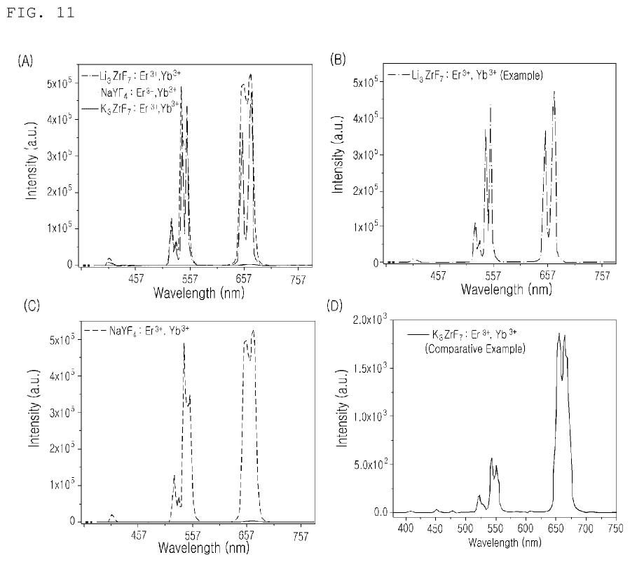

FIG. 10 is a result of measuring a change in brightness of upconversion luminescence over time of upconversion nanoparticles according to an embodiment of the present disclosure.

Referring to FIG. 10, it can be confirmed that the bright yellow UCL gradually disappears over 20 days. Through this, it was confirmed that the degradation time of the upconversion nanoparticles according to Example of the present disclosure was longer than 8 hours, which was the degradation time of the upconversion nanoparticles, Comparative Example (K₃ZrF₇: Yb, Er).

### [Experimental Example 3] Comparison of Upconversion Luminescence Intensity

An experiment was conducted to compare the upconversion luminescence intensity of the upconversion nanoparticles according to Example and Comparative Examples of the present disclosure.

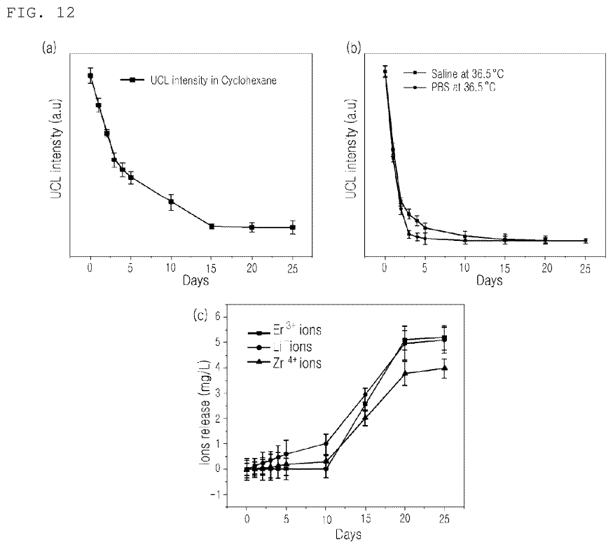

FIG. 11 is a UCL spectrum of upconversion nanoparticles according to an Example and Comparative Example of the present disclosure.

Referring to FIG. 11, it can be confirmed that the UCL intensity of the upconversion nanoparticles of Example is about 300 times higher than that of Comparative Example 1.

### Experimental Example 4

The upconversion nanoparticles of Example were immersed in pure water at room temperature, saline at 36.5° C., and PBS buffer at 36.5° C., respectively, and the UCL intensities associated with the stepwise increased contents of Zr, Li and Er were measured (determined by inductively coupled plasma optical emission spectroscopy (ICP-OES)).

In FIG. 12, (A) and (B) are graphs showing measurement of UCL intensity of upconversion nanoparticles according to an embodiment of the present disclosure in various solvents, and (C) is a graph showing measurement of ion release.

Referring to FIG. 12, it can be firmly confirmed that the biodegradation time of the upconversion nanoparticles of Example is actually 20 days.

### Experimental Example 5

In FIG. 13, (A) and (B) are HRTEM images before and after degradation of upconversion nanoparticles according to an embodiment of the present disclosure, and (C) and (D) are SEAD patterns of upconversion nanoparticles according to an embodiment of the present disclosure.

Referring to FIG. 13, it can be confirmed that the upconversion nanoparticles of Example are reduced to a small size and changed from a crystalline phase to an amorphous phase in an aqueous medium.

### [Experimental Example 6] Characterization of Silica-Coated Upconversion Nanoparticles (Li₃ZrF₇:Er³⁺,Yb³⁺@SiO₂)

The upconversion nanoparticles (Li₃ZrF₇:Er³⁺,Yb³⁺) of Example were coated with a silica (SiO₂) layer that is hydrophilic and suitable for biomedical use through the ORMOSIL method to prepare silica-coated upconversion nanoparticles (Li₃ZrF₇:Er³⁺,Yb³⁺@SiO₂).

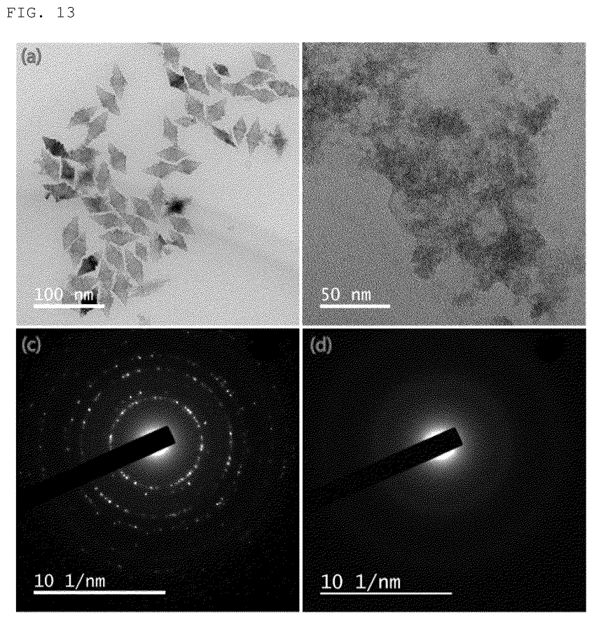

FIG. 14 is an HRTEM image of upconversion nanoparticles coated with silica before and after degradation of the nanoparticles according to an embodiment of the present disclosure.

Referring to (A) in FIG. 14, it can be confirmed that Li₃ZrF₇:Er³⁺,Yb³⁺@SiO₂ maintains the tetragonal nanodendritic morphology without change after the coating process. It also apparently shows the shell with an average thickness of 5±1 nm surrounding the tetragonal nanodendritic morphology resulting from the SiO₂ coating.

Referring to (B) in FIG. 14, it can be confirmed that Li₃ZrF₇:Er³⁺,Yb³⁺@SiO₂ is decomposed in saline at 36.6° C. within days. Through this, it can be confirmed that the synthesized Li₃ZrF₇:Er³⁺,Yb³⁺ nanodendritic crystal is biodegradable even after being coated with silica.

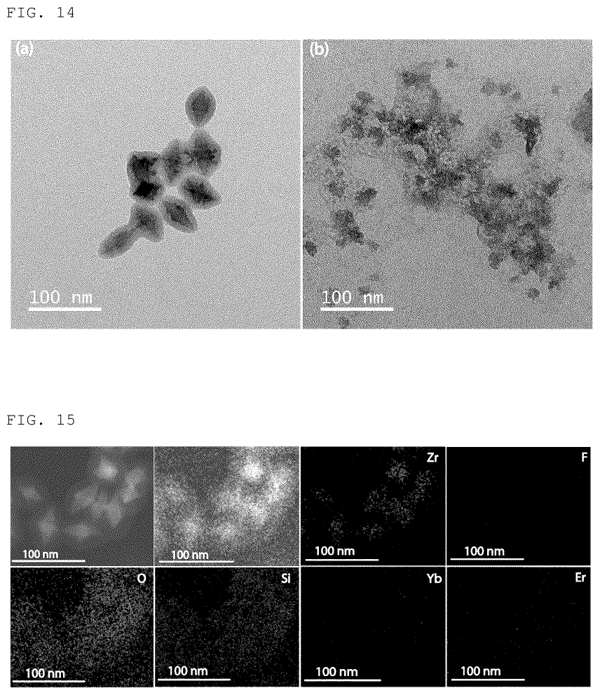

FIG. 15 is a HADDF-STEM image and elemental mapping analysis result of upconversion nanoparticles coated with silica according to an embodiment of the present disclosure.

Referring to FIG. 15, HADDF-STEM image and elemental mapping analysis of upconversion nanoparticles, which has the three-dimensional core Li₃ZrF₇:Er³⁺,Yb³⁺@SiO₂ biodegradable nanodendritic crystal and in which Zr, Yb, Er, Si, O and F are uniformly distributed, are shown, and it can be seen that the core is in nanodendritic morphology. The SiO₂ coating was demonstrated to be reflected from the transparent shell covered the surface of the upconversion nanoparticle, which has the tetragonal Li₃ZrF₇:Er³⁺,Yb³⁺ biodegradable nanodendritic crystal.

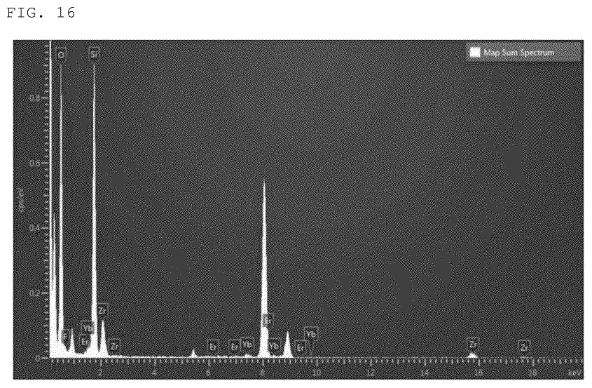

FIG. 16 is an energy dispersive x-ray spectroscopy (EDS) spectrum of nanoparticles coated with silica according to an embodiment of the present disclosure.

Referring to FIG. 16, the energy dispersive X-rays spectroscopy (EDS) was confirmed after coating the upconversion nanoparticles, which had three-dimensional core Li₃ZrF₇:Er³⁺,Yb³⁺@SiO₂ biodegradable nanodendritic crystal, with the silica containing Zr, Yb, Er, Si, O, and F elements.

Unlike last reported biodegradable upconversion that were biodegraded within about 12 hours, the upconversion nanoparticles according to the present disclosure have increased biodegradation period from several hours to several days, and have about 300 times or more upconversion luminescence intensity compared to last reported biodegradable upconversion. Accordingly, the upconversion nanoparticles according to the present disclosure can be applied to various biomedical fields such as bioimaging and used more practically.

In addition, the upconversion nanoparticles according to the present disclosure are essential for biological applications because they have renal filtration efficiency, have a low-toxic degradation pathway for in vivo application, and can be selectively separated only from target sites without degradation at off-target sites. Due to these characteristics, the upconversion nanoparticles according to the present disclosure can be applied to drug delivery systems and used efficiently.

In addition, the upconversion nanoparticles according to the present disclosure are stable in the circulatory system and can be stably used in vivo due to non-toxicity and non-immunogenicity.

However, the effects obtainable herein are not limited to the effects described above, and other effects may exist.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the above detailed description. It shall be understood that all changes or modified embodiments conceived from the meaning and scope of the claims and their equivalents are included in the scope of the present disclosure.

What is claimed is:

1. A upconversion nanoparticle, which is represented by the following Chemical Formula 1 and comprises a nanoparticle doped with lanthanide ion:
Li₃ZrF₇:Ln³⁺ [Chemical Formula 1]
where Ln is a lanthanide element selected from the group consisting of La, Ce, Pr, Nd, Pm, Sm, Eu, Gd, Tb, Dy, Ho, Er, Tm, Yb, Lu, and combinations thereof.

2. The upconversion nanoparticle of claim 1, wherein the nanoparticle doped with the lanthanide ion is the nanoparticle doped with two or more different lanthanide ions.

3. The upconversion nanoparticle of claim 1, wherein the nanoparticle doped with the lanthanide ion includes Li₃ZrF₇:Er³⁺, Yb³⁺.

4. The upconversion nanoparticle of claim 1, wherein the upconversion nanoparticle is biodegradable.

5. The upconversion nanoparticle of claim 1, wherein the upconversion nanoparticle has an upconversion luminescence (UCL) intensity of 5×10⁵ or more in a wavelength range of 400 nm to 700 nm.

6. A method for preparing an upconversion nanoparticle, comprising:
preparing a mixture by dissolving a lithium salt, a zirconium salt, and a lanthanide ion precursor in an organic solvent containing a fatty acid; and
heating the mixture.

7. The method of claim 6, wherein the lanthanide ion precursor comprises two or more different lanthanide ion precursors.

8. The method of claim 7, wherein the lanthanide ion precursor comprises one represented by Chemical Formula 2 below:
Ln(CH₃CO₂)·4H₂O [Chemical Formula 2]
where Ln is La, Ce, Pr, Nd, Pm, Sm, Eu, Gd, Tb, Dy, Ho, Er, Tm, Yb or Lu.

9. The method of claim 6, wherein the lithium salt includes one selected from the group consisting of lithium trifluoroacetate, lithium tetrafluoroborate, lithium hexafluorophosphate, lithium bis(trifluoromethylsulfonyl)imide, lithium trifluoromethane sulfonate, lithium acetate, lithium nitrate, lithium perchlorate, lithium hexafluoroarsinate, lithium bis(pentafluoroethylsulfonyl)imide, lithium dicyanamide, lithium tetrachloroaluminate, lithium hexafluoroantimonate, and combinations thereof.

10. The method of claim 6, wherein the zirconium salt includes one selected from the group consisting of zirconium (IV) acetylacetonate, zirconium acrylate, zirconium (IV) bis(diethyl citrato)dipropoxide, zirconium bromonorbornenelactone carboxylate triacrylate, zirconium (IV) butoxide, zirconium (IV) tert-butoxide, zirconium (IV) carbonate basic, zirconium carboxyethylacrylate (in n-propanol), zirconium (IV) chloride tetrahydrofuran complex, zirconium (IV) ethoxide, zirconium (IV) isopropoxide isopropanol complex, zirconium (IV) propoxide solution (in N-propyl alcohol), zirconium (IV) 2,2,6,6-tetramethyl-3,5-heptanedionate, zirconium (IV) trifluoroacetylacetonate, and combinations thereof, but is not limited thereto.

11. The method of claim 6, wherein the organic solvent includes one selected from the group consisting of 1-octadecene, 1-nonadecene, cis-2-methyl-7-octadecene, 1-heptadecene, 1-hexadecene, 1-pentadecene, 1-tetradecene, 1-tridecene, 1-undecene, 1-dodecene, 1-decene, and combinations thereof.

12. The method of claim 6, wherein the fatty acid includes one selected from the group consisting of oleic acid, elaidic acid, gondoic acid, erucic acid, nervonic acid, eicosadienoic acid, mead acid, and combinations thereof.

13. The method of claim 6, wherein the heating is performed at a temperature range of 100° C. to 350° C.

14. A bioimaging probe comprising the upconversion nanoparticle of claim 1.
